(19) United States
(12) Patent Application Publication
(10) Pub. No.: US 2023/0088270 A1
(43) Pub. Date: Mar. 23, 2023

(54) LID FOR PETRI DISH AND METHOD OF USE

(71) Applicant: Evan Floyd, Forsyth, GA (US)

(72) Inventor: Evan Floyd, Forsyth, GA (US)

(21) Appl. No.: 17/947,912

(22) Filed: Sep. 19, 2022

Related U.S. Application Data
(60) Provisional application No. 63/246,200, filed on Sep. 20, 2021.

(51) Int. Cl. C12M 1/22; C12M 1/00

(52) U.S. Cl. CPC C12M 23/10; C12M 23/38

(57) ABSTRACT

A cover is provided for a Petri dish including a base portion having a continuous peripheral sidewall for holding a layer of a gel medium having a predetermined depth for growing micro-organisms. The cover comprises a top portion having an inner surface, and a depending integral peripheral flange for receiving the sidewall of the base portion in a closed position. A plurality of posts depend orthogonally from the inner surface of the top portion to free distal ends. The posts are sufficiently long such that the free distal ends of the posts extend into the liquid gel medium when the cover is in the closed position. The posts cause an array of wells to form in the surface of the hardened gel medium corresponding to the depth of penetration of the posts into the medium.

### BACKGROUND

A lid for a Petri dish for cultivating bacteria is described and, more particularly, a Petri dish lid which prepares inoculation sites in a solid media for biological testing and analysis, for example, in biofilm inhibition research and other drug susceptibility testing.

Drug susceptibility testing is intended to obtain a treating principle regarding the drug concentration at which growth of the bacteria is inhibited. It is often necessary to determine the efficacy, strength or concentration of drugs or other bacterial growth inhibiting substances, such as anti-biotics. A conventional procedure is to provide a layer of agar nutrient inoculated with a suitable microorganism in a Petri dish, apply a solution of the substance to be tested at a point or area of the agar, and incubate for a period of time. The efficacy of the substance is determined by measuring the size of the area surrounding the point of application of the solution where growth of the microorganism has been inhibited, which occurs in response to diffusion of the drug. A comparison is made based upon an inhibition zone of the bacteria to be tested

There is a need for an improved Petri dish that facilitates the application of the solution of growth-inhibiting substance to be tested to inoculated agar. The new Petri dish should provide distinct plating locations relative to media. Ideally, the new Petri dish can be used to test a plurality of drugs and concentration samples with one Petri dish for conveniently determining, for example, drug susceptibility of bacteria.

### SUMMARY

A cover is provided for a Petri dish including a base portion having a continuous peripheral sidewall for holding a layer of a gel medium having a predetermined depth for growing micro-organisms. The cover comprises a top portion having an inner surface, and a depending integral peripheral flange for receiving the sidewall of the base portion in a closed position. A plurality of posts depend orthogonally from the inner surface of the top portion to free distal ends. The posts are sufficiently long such that the free distal ends of the posts extend into the liquid gel medium when the cover is in the closed position. The posts cause an array of wells to form in the surface of the hardened gel medium corresponding to the depth of penetration of the posts into the medium.

A Petri dish is also provided for holding a layer of a gel medium having a predetermined depth for growing micro-organisms. The dish comprises a base portion having a continuous peripheral sidewall. A cover includes a top portion having an inner surface, and a depending integral peripheral flange for receiving the sidewall of the base portion in a closed position. A plurality of posts depend orthogonally from the inner surface of the top portion to free distal ends. The posts are sufficiently long such that the free distal ends of the posts extend into the liquid gel medium when the cover is in the closed position. The posts cause an array of wells to form in the surface of the hardened gel medium corresponding to the depth of penetration of the posts into the medium.

In one aspect, the top portion is circular for use with a corresponding petri dish base portion. In another aspect, the posts are cylindrical. The posts may have the same or different lengths. In a further aspect, the distal ends of the posts are planar.

In one embodiment, the gel medium comprises an agar nutrient.

A method is also provided for testing the biological activity of substances produced by microorganisms cultured on nutrient gel media. The testing method comprises the steps of providing a test device. The test device includes a base portion having a continuous peripheral sidewall and a cover including a top portion having an inner surface. The cover has a depending integral peripheral flange for receiving the sidewall of the base portion in a closed position. A plurality of posts depend orthogonally from the inner surface of the top portion to free distal ends, the posts being sufficiently long such that the free distal ends of the posts extend into the gel medium when the cover is in the closed position. The method further comprises the steps melting the nutrient gel media, pouring the melted gel media into the base portion, and moving the cover over the base portion such that the peripheral flange receives the sidewall of the base portion in a closed position and the free distal ends of the posts extend into the gel media. The gel media is allowed to harden and the cover removed from the base portion wherein each post forms a well in the hardened gel.

In one aspect, the test method further comprises the step of introducing a test liquid or agent in at least one well, the reactivity of the agent and the sample microorganisms being the subject of investigation.

In another aspect, the test method further comprises the steps of inoculating the gel media by depositing sample microorganisms in at least one well, and incubating the microorganisms for a predetermined time. The wells of the test device are observed for determining the presence or absence of a reaction by the development of areas which indicate biological activity of the microorganisms on the gel in the first well.

In still another aspect, the test method further comprises the step of introducing a test liquid/agent in at least one well or, alternatively, further comprising the step of depositing a reagent in at least one well.

In a further aspect, the test method further comprises the step of depositing an active substance into at least one of the plurality of wells.

### BRIEF DESCRIPTION OF THE DRAWINGS

For a more complete understanding of the lid for a Petri dish and method of use, reference should now be had to the embodiments shown in the accompanying drawings and described below. In the drawings:

FIG. 1 is a top perspective view of an embodiment of a lid for a Petri dish.

FIG. 2 is a bottom perspective view of the Petri dish lid as shown in FIG. 1.

FIG. 3 is a top plan view of the Petri dish lid as shown in FIG. 1.

FIG. 4 is a bottom plan view of the Petri dish lid as shown in FIG. 1.

FIG. 5 is a side elevation view of the Petri dish lid as shown in FIG. 1.

FIG. 6 is a top perspective of a Petri dish in a closed position.

FIG. 7 is an exploded top perspective view of the Petri dish as shown in FIG. 6.

FIG. 8 is a cross-section view of the Petri dish as shown in FIG. 6 taken along line 8-8 of FIG. 4.

FIG. 9 is a top plan view of the Petri dish lid holding solid media formed with six wells by the lid as shown in FIG. 1.

FIG. 10 is a top plan view of the Petri dish lid holding solid media formed with six wells by the lid as shown in FIG. 9 and containing cultured media showing bacterial colony growth in three of the six wells.

### DESCRIPTION

Certain terminology is used herein for convenience only and is not to be taken as a limiting. For example, words such as “upper,” “lower,” “left,” “right,” “horizontal,” “vertical,” “upward,” “downward,” “top” and “bottom” merely describe the configurations shown in the FIGS. Indeed, the components may be oriented in any direction and the terminology, therefore, should be understood as encompassing such variations unless specified otherwise. The words “interior” and “exterior” refer to directions toward and away from, respectively, the geometric center of the core and designated parts thereof. The terminology includes the words specifically mentioned above, derivatives thereof and words of similar import.

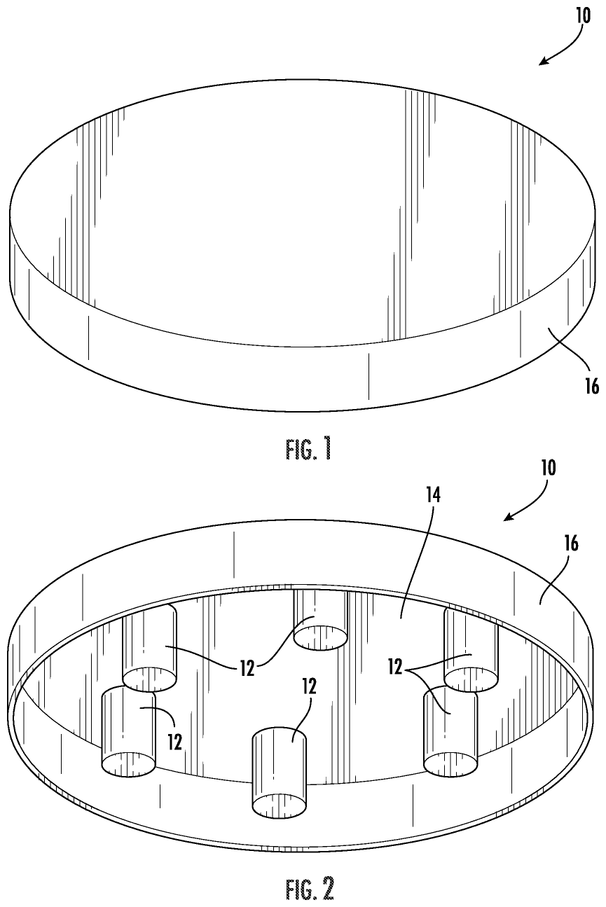

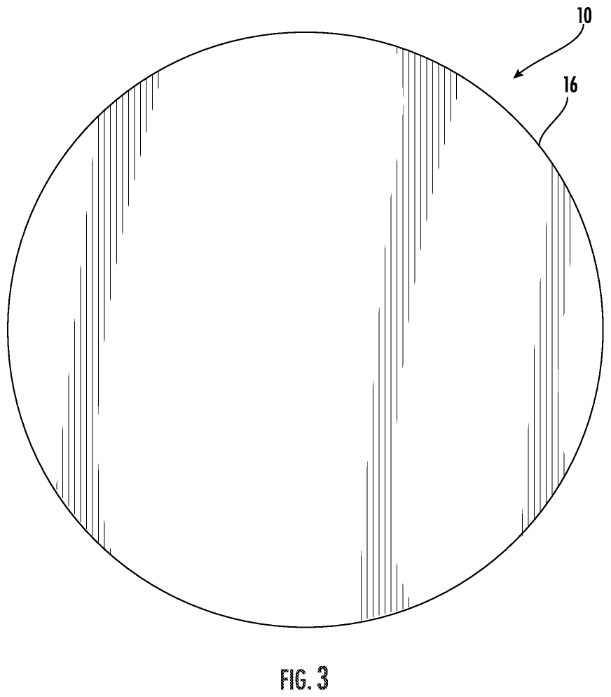

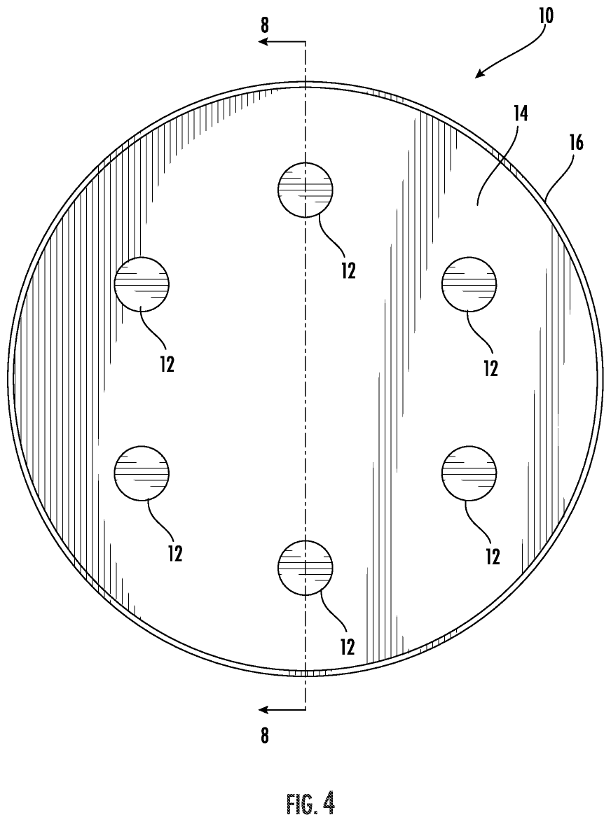

Referring now to the drawings, wherein like reference numerals refer to corresponding or similar elements throughout the several views, an embodiment of a lid for a Petri dish used to create a plurality of open wells in solid media in the Petri dish is shown in FIGS. 1-5 and generally designated at 10. The user plates bacteria and non-antibiotic molecules into the wells for generating growth of a biofilm. Following a period for growth, pixels are measured in a predetermined area defined by the wells to determine the inhibition of the biofilm.

As shown in FIGS. 1-5, the lid 10 of the Petri dish is generally flat and circular as is conventional. The lid 10 may be formed of any suitable material such as metal, synthetic resin, glass, or the like, being preferably a non-porous and relatively non-fragile material capable of withstanding sterilization. The periphery of the lid is formed with an annular projection. Six prongs 12 depend orthogonally inwardly from the inner surface 14 of the lid 10.

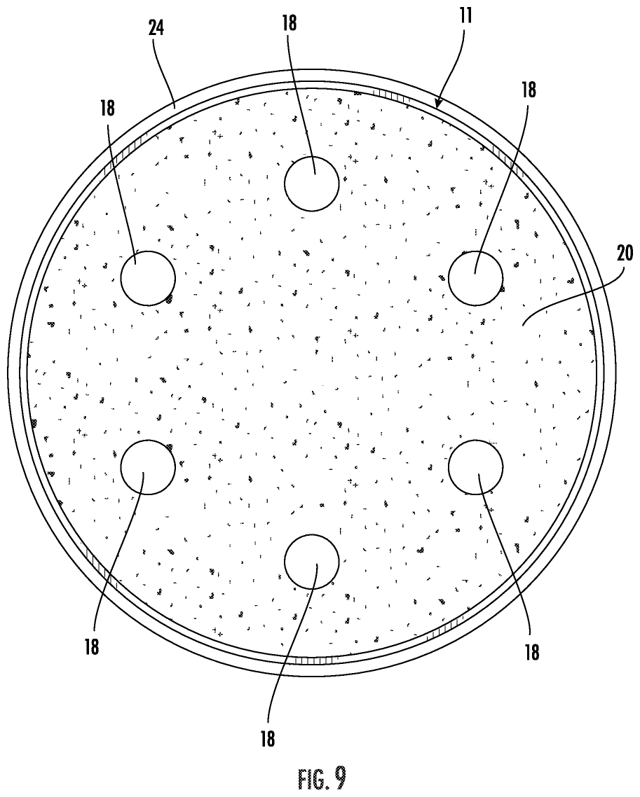

In one embodiment, the Petri dish lid 10 has a diameter of about 92 mm and a thickness of about 1 mm. A peripheral side wall 16 is about 1 mm thick and 5 mm tall. The Petri dish lid 10 may have six prongs 12 depending from the inner surface 14 of the lid. Each prong 12 is cylindrical and about 5 mm in diameter and extends about 10 mm from the inner surface 14 of the lid. Each prong 12 is also about 32 mm from the center of the lid 10, and the prongs 12 are spaced about 60° from one another. It is understood that the number, size and shape and position of the prongs 12 may vary. The prongs 12 are long enough to penetrate the media, but do not reach the bottom of the base 11 of the Petri dish when the lid 10 rests on the base of the Petri dish (FIG. 8).

The Petri dish lid 10 can be made in any suitable manner. In one embodiment, Autodesk Fusion 360 software is used. The above-described dimensions are transferred into a stl file, which is then converted to a comprehendible format using PrusaSlicer software. The sliced file is then printed on a Prusa Mini 3D additive manufacturing printer using Prusa's PLA (polylactic acid) filament. The print is done using the 0.12 mm detail tune for the printer, ensuring exact dimensional specifications is met within a thousandth of a millimeter.

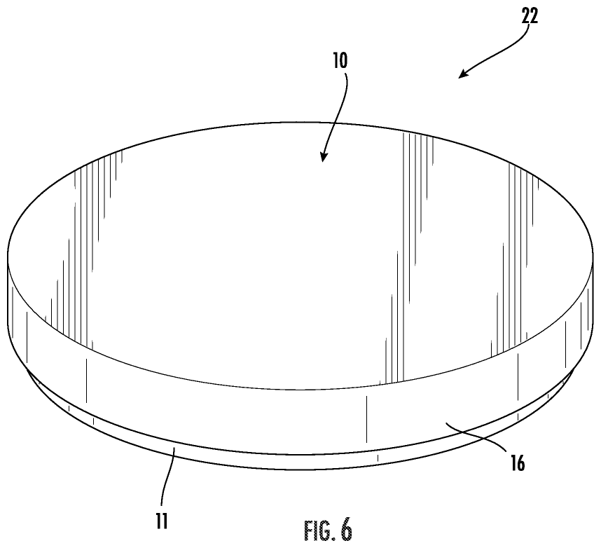

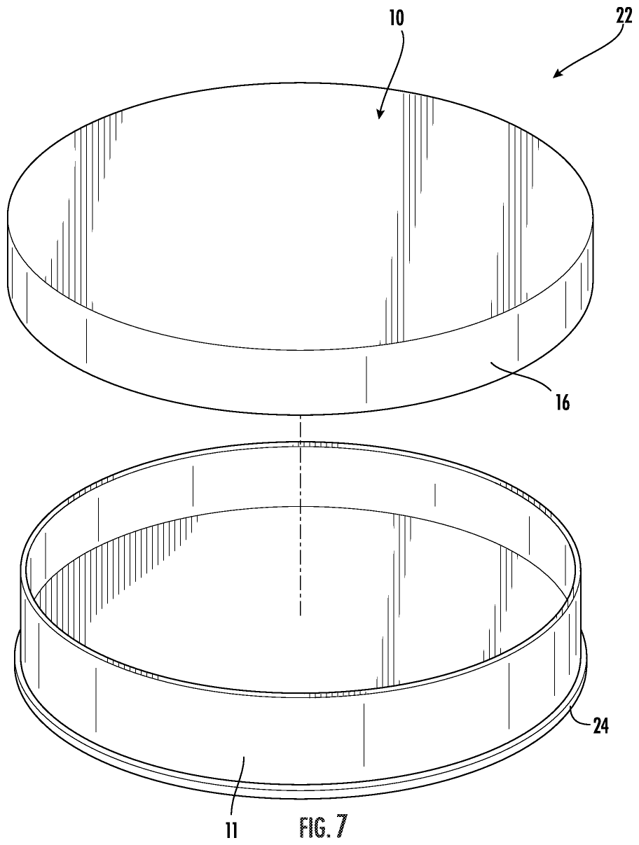

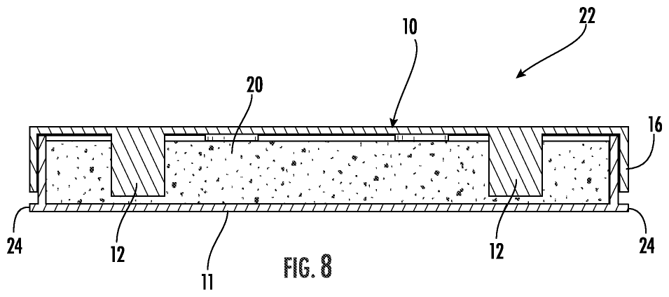

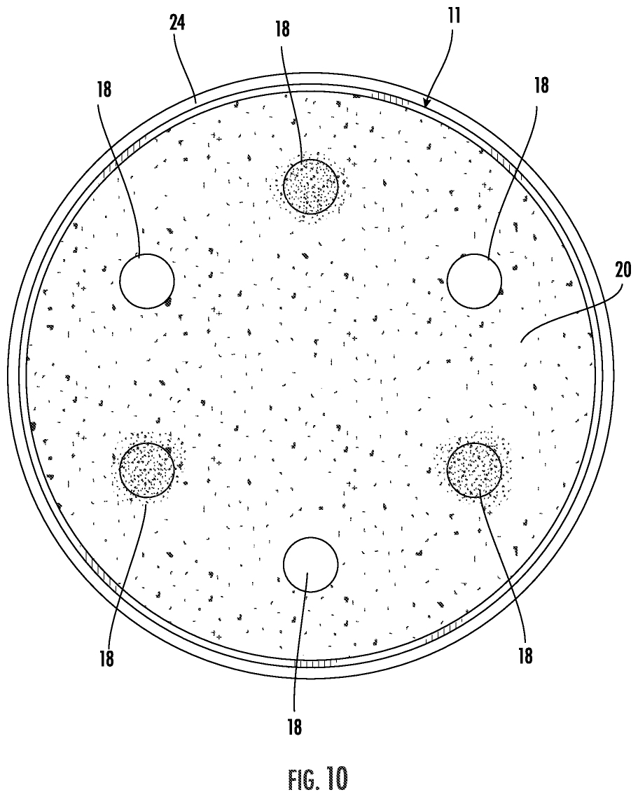

In use, the Petri dish lid 10 as shown in FIGS. 1-5 is provided for a method of molding six, five millimeter diameter wells 18 in gel media 20 within a Petri dish 22 (FIGS. 6 and 7). The method includes the steps of sterilizing a desired number of lids in 70% ethanol, wiping with paper towels and placing under a vent hood so that the ethanol can evaporate. The culture media 20 is sterilized in an autoclave and allowed to cool to a pourable temperature (recommended temperature is 57° C.). A preferred culture media is Congo Red. The dried Petri dishes 22 are filled with the media 20 under the vent hood to about one half to about three quarters full. The Petri dish lid 10 is then seated on the base 11 of the Petri dish 22, or “plate”, for covering the liquid media 20 (FIGS. 6 and 8). The vent hood is closed to prevent contamination, and the media 20 is allowed to solidify for 45 minutes to an hour. Once the media has solidified, the Petri dish lid 10 is carefully pulled upwardly from one side of the lid until the prongs 12 dislodge from the media 20 leaving wells 18 in the media as seen in FIG. 9. The plate 22 can then be Parafilmed or sealed in its original bag and stored at 4° C. to prevent dehydration.

### EXAMPLE 1

To proceed with a testing method for drug susceptibility, a 1:100 diluted overnight of a desired bacterial species is prepared in Tryptic Soy Broth (TSB). Using a 2.0-20.0 μL micropipette, 18.0 μL of the diluted, overnight TSB is aspirated and deposited into an appropriately labeled well 18 in the Petri dish 22. Changing tips of the micropipette with each deposit, this step is repeated on the plate 22 two to five more times depending on species being cultured. Once all the overnight TSB has been deposited, the plate 22 is allowed to sit at room temperature for one hour to allow for the bacterial matrix to settle to the bottom of the wells 18. Next, a 0.2-2.0 μL micropipette is used to add 2.0 μL of 1% by volume of a DMSO solution to one or two of the wells 18, changing tips each time. Then 2.00 μL of a competitive inhibition drug is dispensed into the remaining wells 18.

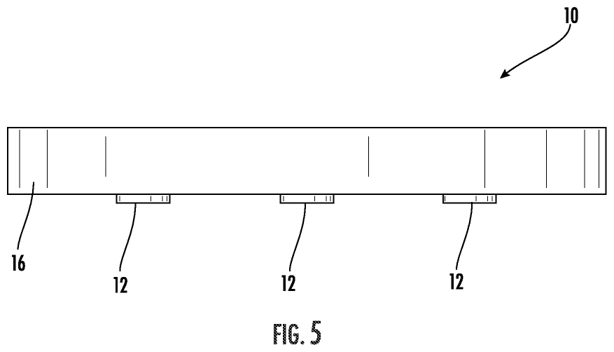

The plates 22 are placed into an incubator at 37° C. for 24 hours. After incubation, photographs of the bottom surface 24 of the plates 22 is taken being sure that the bacteria clouds are clearly visible (FIG. 5). ImageJ software is used. The software is available for downloading from the NIH website where instructions and information of how to use the program may be found. Using the ImageJ software, the images are imported, transferred to 24-bit greyscale, and a rectangular select tool is used to make a square which has a height and length of 180 pixels. The mean grey value is measured. The values of the separate DMSO and drug trial wells 18 are compared to obtain a figure of inhibition or acceleration.

Applicant notes that while the NIH website states, “Mean gray values are a ratio of light to dark pixels.”, this is not the case. Gray values are listed from 0-255 (0 being black and 255 being pure white), and calculating the mean value would be adding all the values of each individual pixel and then dividing by the total pixel area. Taking this into consideration, alterations to the mathematical quantification method are necessary. Specifically, after importing the image into a word document, all the saturation is filtered out (saturation 0% filter) and then the image is transformed to a black and white image. The image is scaled up to 100% of its usual size (right click image>image size). The image is then imported to ImageJ software. Once in ImageJ, a 400×400 pixel area (160,000 pix²) is made around a colony of choice, then right click and select “calculate”. A window then populates the screen showing the total pixel area, the mean gray value, minimum gray value, and the maximum gray value. To obtain the total black pixels (pixels of the colony) the mean gray value is multiplied by 160,000, the result divided by 255, and then that number is subtracted from 160,000. This methodology accounts for every white pixel in the whole population and removes them from the total, leaving only black pixels in the total.

Once each black pixel for each colony on a plate 22 is recognized, a comparison is made between control colonies and drug treatment colonies. Table 1 lists calculated percent inhibition based on the gray values.

TABLE 1

| | | Average of Drug | | | |
|---|---|---|---|---|---|
| | | Black Pixels for | DMSO to | % Inhibition | Total Average |
| Plate | Drug | Listed Plate | Drug Ratio | Detected | Inhibition |
| Jul. 22, 2021; Plate | Tyr 64 | 24837.64706 | 0.450448913 | 54.95510873 | 33.92007939 |
| 1; B. sub | | | | | |
| Jul. 22, 2021; Plate 1; B. sub Report: Based on the calculations in this sheet, | | | | | |
| this plate in particular showed 54.96% inhibition of B. subtilis biofilm. | | | | | |
| Jul. 22, 2021; Plate | Tyr 64 | 25477.96078 | 0.867731595 | 13.22684047 | |
| 2; B. sub | | | | | |
| Jul. 22, 2021; Plate 2; B. sub Report: based on the calculations in this sheet, | | | | | |
| this plate showed 13.23% inhibition of B. subtilis biofilm | | | | | |
| Jul. 22, 2021; Plate | Tyr 64 | 13820.86275 | 0.691390188 | 30.8609812 | |
| 3; B. sub | | | | | |
| Jul. 22, 2021; Plate 3; B. sub Report: Based on the calculations in this sheet, | | | | | |
| this plate showed 30.86% biofilm inhibition in B. subtilis. | | | | | |
| Jul. 22, 2021; Plate | Tyr 64 | 24576.94118 | 0.633626128 | 36.63738717 | |
| 4; B. sub | | | | | |

Jul. 22, 2021; Plate 4; B. sub Report: based on the calculations in this sheet,
this plate showed 36.64% biofilm inhibition in B. subtilis.

The new Petri dish lid 10 and method of use has many advantages, including determining discrete plating locations for subsequent biological testing and analysis. This design allows several types or concentrations of growth inhibiting drugs to be used separately in a well 18 of one Petri dish 22. For example, each well may contain the same type of drug, but in different concentrations. Measuring and comparing the physically isolated colonies per fixed area is simple to complete and enables the number of live bacteria contained in the well 18 to be assessed. Thus, in addition to drug susceptibility in accordance with conventional standards, patterns of the sensitivity of the bacteria in relation to each drug at its various concentrations stages can be easily and rapidly obtained.

I claim:

1. A cover for a Petri dish including a base portion having a continuous peripheral sidewall for holding a layer of a gel medium having a predetermined depth for growing micro-organisms, the cover comprising:
a top portion having an inner surface, and a depending integral peripheral flange for receiving the sidewall of the base portion in a closed position; and
a plurality of posts depending orthogonally from the inner surface of the of the top portion to free distal ends, the posts being sufficiently long such that the free distal ends of the posts extend into the liquid gel medium when the cover is in the closed position,
wherein the posts cause an array of wells to form in the surface of the hardened gel medium corresponding to the depth of penetration of the posts into the medium.

2. The Petri dish cover as recited in claim 1, wherein the top portion is circular for use with a corresponding petri dish base portion.

3. The Petri dish cover as recited in claim 1, wherein the gel medium comprises an agar nutrient.

4. The Petri dish cover as recited in claim 1, wherein the posts are cylindrical.

5. The Petri dish cover as recited in claim 1, wherein the posts have the same or different lengths.

6. The Petri dish as recited in claim 1, wherein the distal ends of the posts are planar.

7. A Petri dish for holding a layer of a gel medium having a predetermined depth for growing micro-organisms, the dish comprising:
a base portion having a continuous peripheral sidewall;
a cover including a top portion having an inner surface, and a depending integral peripheral flange for receiving the sidewall of the base portion in a closed position; and
a plurality of posts depending orthogonally from the inner surface of the top portion to free distal ends, the posts being sufficiently long such that the free distal ends of the posts extend into the liquid gel medium when the cover is in the closed position,
wherein the posts cause an array of wells to form in the surface of the hardened gel medium corresponding to the depth of penetration of the posts into the medium.

8. The Petri dish as recited in claim 7, wherein the top portion is circular for use with a corresponding petri dish base portion.

9. The Petri dish as recited in claim 7, wherein the gel medium comprises an agar nutrient.

10. The Petri dish as recited in claim 7, wherein the posts are cylindrical.

11. The Petri dish cover as recited in claim 7, wherein the posts have the same or different lengths.

12. The Petri dish as recited in claim 7, wherein the distal ends of the posts are planar.

13. A method for testing the biological activity of substances produced by microorganisms cultured on nutrient gel media, the testing method comprising the steps of:
providing a test device including
a base portion having a continuous peripheral sidewall,
a cover including a top portion having an inner surface, and a depending integral peripheral flange for receiving the sidewall of the base portion in a closed position, and
a plurality of posts depending orthogonally from the inner surface of the top portion to free distal ends, the posts being sufficiently long such that the free distal ends of the posts extend into the gel medium when the cover is in the closed position;
melting the nutrient gel media and pouring the melted gel media into the base portion;
moving the cover over the base portion such that the peripheral flange receives the sidewall of the base portion in a closed position and the free distal ends of the posts extend into the gel media;
allowing the gel media to harden; and
removing the cover from the base portion wherein each post forms a well in the hardened gel.

14. The testing method as recited in claim 13, further comprising the step of introducing a test liquid in at least one well, the reactivity of the agent and the sample microorganisms being the subject of investigation,

15. The testing method as recited in claim 14, further comprising the steps of
inoculating the gel media by depositing sample microorganisms in at least one well; and
incubating microorganism for a predetermined time.

16. The testing method as recited in claim 15, further comprising the step of observing the wells for determining the presence or absence of a reaction by the development of areas which indicate biological activity of the microorganisms on the gel in the first well.

17. The testing method as recited in claim 13, further comprising the step of depositing a reagent in at least one well.

18. The testing method as recited in claim 13, further comprising the step of depositing an active substance into at least one of the plurality of wells.
